(19) United States
(12) Patent Application Publication
(10) Pub. No.: US 2005/0177063 A1
(43) Pub. Date: Aug. 11, 2005

(54) Hoof Temperature sensor

(76) Inventor: Jeffrey A. Winnie, Molalla, OR (US)

Correspondence Address: IPSOLON LLP, 805 SW BROADWAY, #2740, PORTLAND, OR, 97205, US

(21) Appl. No.: 10/775,651

(22) Filed: Feb. 9, 2004

(51) Int. Cl. A61B 5/00; G01K 11/00

(52) U.S. Cl. 600/549; 374/162

(57) ABSTRACT

A hoof temperature sensor comprises temperature sensitive strips or modules affixed to a hoof that provide a visual indication of the hoof temperature. The temperature sensitive modules may be configured for indicating temperature according to the color of the module or a portion of it, by numeric values printed on the strip, or combinations of color and numeric values. The visual indication of temperature may be either reversible, irreversible, or a combination of both.

### FIELD OF THE INVENTION

This invention relates generally to the field of animal health and more particularly to an apparatus and method for determining and monitoring the temperature of a hoof, especially a horse's hoof.

### BACKGROUND

Most horse owners and professionals such as farriers and veterinarians who work with horses on a daily basis know that the temperature of a horse's hoof can be an accurate indicator of the animal's health. In most instances a horse's hoof feels slightly warm to the touch; this is a normal condition. However, a hoof that feels either unusually warm or unusually cold to the touch may be an indication of a health problem of some kind. As such, most people who work with horses monitor the temperature of their horse's hooves at regular intervals.

One typical way of monitoring hoof temperature is to feel the hoof with a hand. While this is a time-tested way to identify potential health problems, it is very subjective and is not a reliable indicator of hoof temperature.

Broadly stated, laminitis is an inflammation of the laminae, which is a tissue on the interior of the hoof. Although there are numerous conditions that may result in such inflammation, in common usage, the word laminitis is used to describe a range of conditions that can result in equine lameness, even though some of those conditions are technically not laminitis. Thus, while the word laminitis is on occasion used to describe conditions that are not manifest by inflammation of the laminae, most of the conditions that are called laminitis are accompanied by elevations in hoof temperature, and hoof temperature may thus be a diagnostic indicator of an adverse physical condition.

There are many, many conditions that may cause or contribute to laminitis (or to conditions that are often referred to as laminitis). To cite just a few examples, laminitis may be caused by any of the following:
dietary origins; overweight and under exercised animals;
concussive origins; excessive work on hard surfaces resulting in trauma;
acute illness; may be accompanied by sudden elevated temperature;
production of toxins from kidney or liver disease or in some instances during foaling;
rapid changes in environmental conditions and temperature extremes such as severe cold during winters;
illnesses that are accompanied by toxemia; bacterial infections;
Cushing's Disease;
weight bearing origins; often seen when an animal is lame in one leg and the animal thus spends an excessive amount of time standing on another leg;
stress-induced; extreme stress in some animals may induce laminitis;
drug induced laminitis; and
abscesses and other bacterial infections from whatever source (not technically an inflammation of the laminae but often called laminitis).

Regardless of the source of the problem that is causing laminitis, as noted, one symptom that is commonly associated with it is elevated or depressed hoof temperature. Elevated hoof temperature is typically a result of digital vasodilation in the hoof. On the other hand low hoof temperature is typically the result of digital vasoconstriction. And again, while feeling the horse's hoof with one's hand is one way of detecting an unusual condition, it is not reliable. Most people are not experienced enough to discern a slight difference in hoof temperature, and thus may not be able to identify, or may misidentify, a potentially problematic condition. Moreover, the temperature of the outer surface hoof may be affected by existing environmental conditions (such as extreme cold), and may mask actual elevations in hoof temperature and associated detection of the temperature with the human hand.

There is a need therefore for apparatus that aids in measuring and monitoring hoof temperature, particularly in horses, in order to assist in determining if an adverse health indication is present.

### SUMMARY OF THE INVENTION

The present invention addresses the need for apparatus that provide an indication of hoof temperature. The invention comprises temperature sensitive strips or modules affixed to a hoof that provide a visual indication of the hoof temperature. The temperature sensitive strips may be configured for indicating temperature by the color of the strip, by numeric values printed on the strip, or combinations of color and numeric values. The visual indication of temperature may be either reversible, irreversible, or a combination of both.

### BRIEF DESCRIPTION OF THE DRAWINGS

The invention will be better understood and its numerous objects and advantages will be apparent by reference to the following detailed description of the invention when taken in conjunction with the following drawings.

FIG. 1 is a perspective view of the lateral portion of a horse's front hoof showing a first preferred embodiment of the illustrated invention.

FIG. 2 is a perspective view of the medial portion of the horse's front hoof shown in FIG. 1.

FIG. 3 is a perspective view of the frontal portion of the horse's front hoof shown in FIG. 1.

FIG. 4 is a plan view of the back side of one illustrated temperature sensing strip according to the invention, showing the strip backing material partially cut away.

FIG. 5 is a plan view of an alternate form of a temperature sensing strip according to the invention.

FIG. 6 is a perspective view of the frontal portion of the horse's front hoof shown in FIG. 1 with interconnecting bands between the temperature sensing portions removed.

FIG. 7 is a perspective view of the frontal portion of a horse's front hoof shown with the alternative temperature sensing strip shown in FIG. 5, and shown with the interconnecting bands between the temperature sensing portions removed.

FIG. 8 is a perspective view of the lateral portion of a horse's front hoof showing an alternative embodiment of the illustrated invention.

### DETAILED DESCRIPTION OF THE PREFERRED EMBODIMENTS

A preferred embodiment of the illustrated invention is shown in the attached drawings and is described herein. It will be understood that although the invention is described and illustrated with respect to use of the invention on a front hoof of a horse, the invention is not limited to use on the front hoof, but instead may beneficially be used on any and all hooves. Moreover, the invention is not limited to use with horses, but may be used with any hoofed animal.

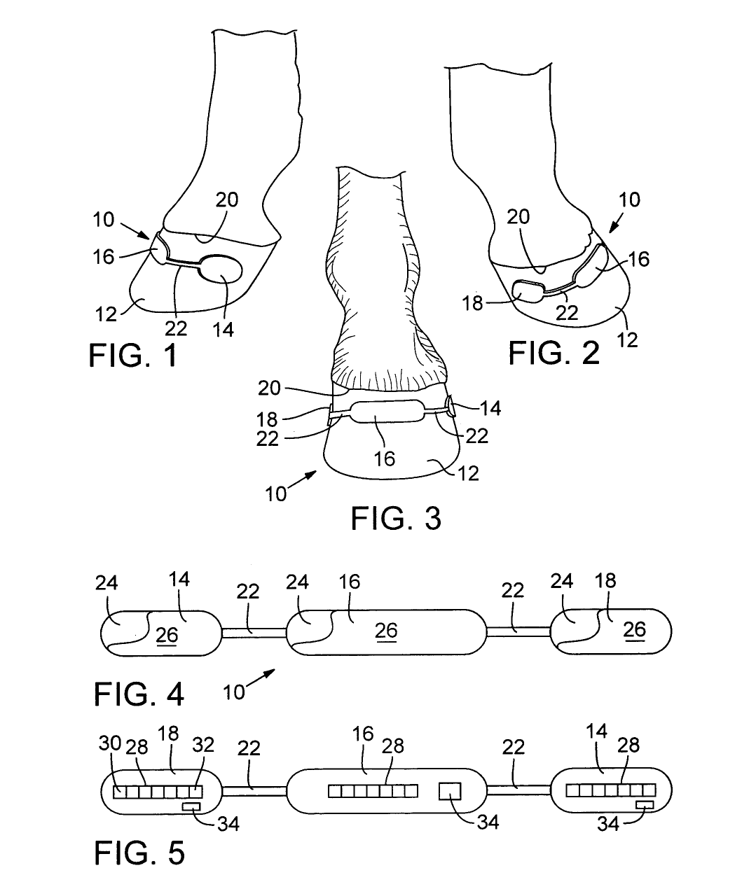

With reference to FIGS. 1, 2 and 3, which show the lateral, medial and frontal sides respectively of the hoof 12, the illustrated invention comprises a hoof temperature-sensing apparatus 10 comprising one or more temperature-sensing modules 14, 16 and 18, applied and attached to hoof 12 below the coronet band 20. There are three temperature-sensing modules shown in the FIGS. 1, 2 and 3, labeled with reference numbers 14, 16 and 18, and each is attached to one “side” of the hoof. Thus, module 14 is attached to the portion of the hoof that is referred to as the lateral portion. Module 16 is attached to the frontal portion of the hoof, and module 18 is attached to the medial portion. Each module 14, 16 and 18 is shown in FIGS. 1 and 2 as being interconnected to the adjacent module by an interconnecting band 22. As detailed below, the interconnecting bands 22 assist in locating and applying the temperature-sensing apparatus 10 to the hoof. However, the interconnecting bands are optional, and when used, may be removed if desired after the apparatus 10 is applied. Furthermore, although there are three modules illustrated, the invention is not limited to three modules per hoof, as more or less than three may be used. However, in most instances three modules, placed as shown in the drawings, provide sufficient information regarding hoof temperature.

With reference now to FIG. 4, temperature-sensing apparatus 10 comprises a flexible temperature-sensitive indicator strip. In the illustrated embodiment, apparatus 10 includes three separate temperature-sensing portions or modules 14, 16 and 18, which are interconnected by bands 22. The bands 22 are not temperature-sensitive, but instead help hold apparatus 10 together during application to a horse's hoof. Modules 14, 16 and 18 are shown in FIGS. 1, 2 and 3 as being generally rectangular in shape with rounded corners, but may be most any shape.

The temperature-sensing modules 14, 16 and 18 preferably include temperature sensitive substances that provide a visual indication of changes in temperature or which illuminate temperature scale indicators (as described more fully below) to thereby provide a visual display of the temperature of the hoof. The modules 14, 16 and 18 measure the temperature of the hoof in the areas of the hoof that the modules cover, and the area that generally surrounds the modules. As such, where three modules are used as shown in FIGS. 1, 2 and 3 on the lateral, medial and frontal portions of the hoof, a good approximation is obtained for the temperature of the entire hoof. Moreover, the individual modules provide an indication of the temperature at each spot where the modules are applied to the hoof.

The temperature-sensitive modules 14, 16 and 18 are, while capable of taking on numerous forms and having numerous capabilities as described herein, implemented according to conventional technologies for temperature-sensitive flexible devices, such as liquid crystal composition and the like. Furthermore, the temperature sensing modules may be of any size or shape, may sense any desired temperatures or temperature ranges, and may include a temperature scale arranged in any fashion and having any type of indicators (e.g., alphanumeric or other characters or colorimetric) representing any temperature intervals or other information for any desired temperature range. Alternatively, the temperature sensing module may be configured with or without a temperature scale and may change to various colors, such as red, green and blue, based on the temperature of the hoof to indicate that the temperature is above, within or below a specified predetermined range. Moreover, the temperature-sensing modules may be reversible, in which case the temperature of the hoof is continuously displayed, or may be irreversible, in which case the color (for example) of the module changes if the temperature of the hoof exceeds a predetermined threshold temperature or goes outside of a temperature range, and does not change color back to the original color even when the temperature of the hoof goes back below the threshold temperature value. The modules also may comprise any combination of reversible and irreversible functionality in, for example, different locations on the modules.

Each temperature-sensing module 10 is configured for exhibiting a visual indication of hoof temperature or a change in hoof temperature according to predetermined criteria. For example, if the animal to which the module is to be applied is a horse, then the module will be configured for responding to predetermined temperatures typical of a horse. Because the normal temperature of other animal species may be different from a horse, the predetermined temperature values and/or ranges at which the temperature sensitive modules exhibit a visual indication temperature may be different from the values used with a module intended for equine use. Thus, the specific temperature or temperature ranges at which the modules react will vary according to predetermine criteria such as the type of animal, the conditions where the animal lives, etc.

Temperature-sensing apparatus 10, including modules 14, 16 and 18 preferably includes an adhesive backing 24 that is covered by a removable protective layer 26. The removable layer 26 covers the adhesive backing 24 to protect the adhesive from contamination during storage. Removable layer 26 is shown partially cut away in FIG. 4. Adhesive backing 24 is optional because the modules are themselves glued or otherwise affixed to the hoof as described below, but the adhesive backing is beneficial because it aids in correctly positioning the apparatus on the hoof while it is being placed on the hoof.

Each temperature-sensing module, such as modules 14,16 and 18, incorporates a temperature-sensitive substance that provides a visual indication of the temperature of the subject animal's hoof. As shown in FIG. 5, the modules may be provided with a temperature scale indicator 28, which may be calibrated according to any desired temperature range and with any desired increments between divisions within the scale indicator. Each division in scale indicator 28 may be associated with a numeric value printed onto the module. The divisions in scale indicator 28 may further include varying color indicators such as are provided with thermochromic materials incorporated into the modules. For example, with reference to scale indicator 28 associated with temperature sensitive module 18 in FIG. 5, the color of division 30 may be a color typically associated with normal temperatures—e.g., green—whereas the color of division 32 at the opposite end of the scale indicator may be a color typically associated with abnormal or excessive temperatures—e.g., red. The scale indicator may thus be calibrated so that normal hoof temperatures are manifest with a green color, and abnormal hoof temperatures with a red color. Numeric values corresponding to the temperature of any given division may additionally be printed above that division so that a numeric value of temperature is correlated with a color.

In other instances it may be desirable to have the temperature-sensitive module exhibit an absolute temperature condition. For example, if the temperature of the hoof rises above a predetermined value, then the module turns red. If the temperature is below the predetermined value, then the module remains green. In this case the module may be reversible or irreversible. If reversible, then the color may change from green to red and back to green, and so on, thereby providing a visual indication of hoof temperature on a real-time basis. If the module is irreversible, then if the hoof temperature exceeds a predetermined threshold temperature, then the color changes (for example, from green to red) and remains that color regardless of whether the actual hoof temperature decreases to below the threshold value. The reversible and irreversible attributes may be combined in one module or between modules. As an example, approximately 1/2 of a module 14 may be configured for reversible color indication, and the other ½ may be irreversible. Using this type of a module, the user knows the real-time temperature of the hoof, but also has an indication that the hoof temperature has at some point risen above (or dropped below) the predetermined temperature threshold values built into the module.

The reversible/irreversible structure described above may also be applied to the temperature scale indicators 28. For example, a module 14 that includes a scale indicator 28 may also include a separate threshold temperature indicator 34, which comprises a temperature indicator that irreversibly changes color if a predetermined threshold is exceeded. Thus, indicator 34 may normally be green. The indicator 34 turns to red if the hoof temperature exceeds the predetermined threshold value for that indicator. At the same time, the real time temperature is displayed visually on scale indicator 28. Thus, if the indicator 34 is red, but the temperature on scale indicator 28 reads a normal temperature, the horse owner knows that the hoof temperature has at some point risen above what is considered normal, but is presently within a normal range.

Although a horse's hooves normally are subjected to significant abrasion, apparatus 10 is applied to a hoof 12 in a manner intended to ensure that the apparatus remains in place until intentionally removed. A hoof has three basic layers: the outermost layer or stratum externum, a middle layer or stratum medium, and innermost layer called the stratum internum. The stratum externum is typically only a few millimeters thick and comprises horn tissue composed of keratinized epithelial cells arranged in tubules that run perpendicular to the ground surface of the wall. These epithelial calls are produced by the perioplic dermis, which lies directly proximal to the coronary dermis near the coronary band 20, and the layer of cells is often called the periople. To apply apparatus 10 to the hoof, the periople layer adjacent the coronet band 20 is first abraded as with a fine grit sanding block or brush having moderately stiff bristles. Care is taken to only abrade the periople slightly. The abraded surface of the hoof is then cleaned thoroughly with a suitable cleaner—warm soapy water will suffice, as will common solvents such as acetone. The cleaned surface is allowed to dry completely. If apparatus 10 includes an adhesive backing 24 as described above, the removable layer 26 is next removed from the apparatus to expose the adhesive backing. The apparatus is then pressed against the hoof as illustrated with the individual temperature-sensitive modules (e.g., 14, 16 and 18) located in the desired positions on the hoof (e.g., laterally, frontally and medially, respectively)—the adhesive backing 24 holds the apparatus 10 in place. The modules are preferably located in proximity to the coronet band 20, as that position provides a reliable measure of the hoof temperature, although other locations may be appropriate as indicated. As an example, in an animal that is suspected of having a localized abscess, the module may be positioned over or near the location of the abscess. A suitable adhesive such as an epoxy is then brushed over the entire apparatus 10 and around the periphery of each module 14, 16 and 18 and is allowed to dry. The glue helps keep the apparatus in place even when subjected to normal horse activity. It will be appreciated that the modules may be affixed to the hoof with other appropriate adhesives.

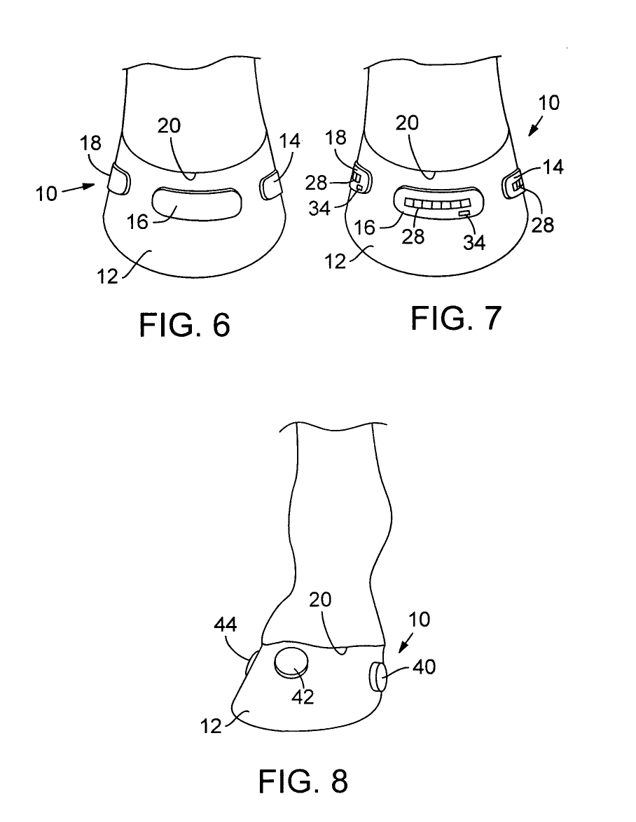

Interconnecting bands 22 may be removed if desired by cutting the bands near where the bands join the individual modules. With reference now to FIG. 6, a hoof 12 is shown with apparatus 10 affixed and interconnecting bands 22 removed. Apparatus 10 is glued to hoof 12 with module 14 positioned laterally, module 16 frontally, and module 18 medially. With three modules positioned in this way the horse owner has a quick and ready visual indication of the hoof temperature at these three locations. More than three modules or less than three modules may be applied as indicated. Furthermore, in the embodiment illustrated in FIG. 6, the modules are of the types described above that comprise color sensitive materials, either reversible or irreversible. The apparatus illustrated in FIG. 7 is applied and affixed to hoof 12 in the same manner described above, except that the modules 14, 16 and 18 are of the type that include temperature scale divisions 28 and threshold temperature indicators 34.

### Alternative Illustrated Embodiments

One possible alternative embodiment of apparatus 10 is shown in FIG. 8 and comprises three separate temperature sensitive modules 40, 42 and 44, positioned laterally, frontally and medially, respectively, on hoof 12. Modules 40, 42 and 44 are round, and may be of any of the types described above.

While the present invention has been described in terms of a preferred embodiment, it will be appreciated by one of ordinary skill that the spirit and scope of the invention is not limited to those embodiments, but extend to the various modifications and equivalents as defined in the appended claims.

1. Apparatus for indicating the temperature of a hoof, comprising:
a temperature sensitive module capable of providing a visual indication of the temperature of a hoof to which said module is affixed.

2. The apparatus according to claim 1 wherein said visual indication of hoof temperature is indicated by a color.

3. The apparatus according to claim 1 wherein said visual indication of hoof temperature correlates to a numeric value.

4. The apparatus according to claim 3 wherein said numeric value is correlated to a color.

5. The apparatus according to claim 1 further comprising plural individual temperature sensing modules, each of said modules capable of providing a visual indication of the temperature of a hoof.

6. The apparatus according to claim 5 including at least 3 individual temperature sensing modules.

7. The apparatus according to claim 2 wherein said module is configured for changing color when a predetermined temperature value has been exceeded.

8. The apparatus according to claim 7 wherein said color change is reversible.

9. The apparatus according to claim 7 wherein said color change is irreversible.

10. The apparatus according to claim 1 including means for adhering said module to a hoof.

11. Apparatus for indicating the temperature of a hoof, comprising:
temperature sensitive module means for providing a visual indication of the temperature of a hoof to which said temperature sensitive module means is affixed.

12. The apparatus according to claim 11 wherein said temperature sensitive module means further comprises a temperature sensitive strip configured for changing color in response to a change in temperature in said hoof.

13. The apparatus according to claim 12 wherein said temperature sensitive module means further comprises means for determining a numeric value for the temperature of said hoof.

14. The apparatus according to claim 11 including plural temperature sensitive module means, each for providing a visual indication of the temperature of a hoof to which said temperature sensitive module means is affixed.

15. The apparatus according to claim 11 wherein said temperature sensitive module means is configured for changing color when a predetermined temperature value has been exceeded.

16. A method for monitoring the temperature of an animal's hoof, comprising the steps of adhering a temperature sensitive module to a hoof, wherein said module is configured for providing a visual indication of the temperature of said hoof.

17. The method according to claim 16 including the steps of abrading the surface if said hoof, cleaning said abraded hoof, allowing said cleaned hoof to dry, and adhering said module to said hoof.

18. The method according to claim 17 wherein said adhering step includes the step of exposing an adhesive backing on said module and applying said module to said hoof.

19. The method according to claim 18 including applying a glue to said module.

20. The method according to claim 16 wherein said animal is a horse and said module is applied to the coronet band.
